(19) United States
(12) Patent Application Publication
(10) Pub. No.: US 2025/0141169 A1
(43) Pub. Date: May 1, 2025

(54) SLIP RING ASSEMBLY, AND MEDICAL DEVICE HAVING SLIP RING ASSEMBLY

(71) Applicant: Shanghai United Imaging Healthcare Co., Ltd., Shanghai (CN)

(72) Inventors: Jianxiong Zou, Shanghai (CN); Feichao Fu, Shanghai (CN); Peng Wang, Shanghai (CN); Cheng Ni, Shanghai (CN); Tuota Xing, Shanghai (CN)

(21) Appl. No.: 19/004,430

(22) Filed: Dec. 29, 2024

Related U.S. Application Data
(63) Continuation of application No. PCT/CN2022/102544, filed on Jun. 29, 2022.

(51) Int. Cl. H01R 39/10

(52) U.S. Cl. CPC H01R 39/10; H01R 2201/12

(57) ABSTRACT

A slip ring assembly includes a base body, at least one first conductor, and at least one second conductor. The base body is configured to have an annular shape and adapted to be made of an insulating material, and the base body includes a first side and a second side opposite to the first side along an axial direction of the base body. The first conductor is configured to have an annular shape and adapted to be fixed on the first side or the second side. The second conductor is configured to have an annular shape and adapted to be fixed on the first side or the second side. The first conductor and the second conductor are electrically insulated, and a projection of the first conductor and a projection of the second conductor along the axial direction of the base body at least partially overlap.

### CROSS-REFERENCE TO RELATED APPLICATIONS

The present application is a continuation application of PCT Patent Application No. PCT/CN2022/102544, filed on Jun. 29, 2022, and entitled “SLIP RING ASSEMBLY, AND MEDICAL DEVICE HAVING SLIP RING ASSEMBLY”, the entire content of which is incorporated herein by reference.

### TECHNICAL FIELD

The present application relates to the field of magnetic resonance medical technology, and in particular to a slip ring assembly, and a medical device having the slip ring assembly.

### BACKGROUND

In modern medicine, imaging equipment is usually used to obtain images of patients to make it easy for a doctor to diagnose or to treat patients by using treatment equipment. For example, magnetic resonance imaging devices are used to obtain magnetic resonance images of tissues of patients, such as tumor patients, to clearly display the conditions of the diseased site and surrounding tissues, so that the diseased tissue can be accurately located. Further, a precise radiation therapy is performed by using the treatment equipment (such as an electronic linear accelerator) based on magnetic resonance images. Magnetic resonance and the treatment device may be used in combination, and the slip ring assembly may be used as a power supply medium and a communication medium of the treatment device. When the slip ring assembly is powered on, the magnetic field generated by the current flowing through the slip ring assembly will interfere with the central magnetic field of the MRI device, thus resulting in a lower accuracy of MRI.

### SUMMARY

Based on this, in order to address the above technical problems, it is necessary to provide a slip ring assembly and a medical device having the slip ring assembly, which can effectively reduce the interference of a magnetic field generated by a current flowing through the slip ring assembly in the central magnetic field of the MRI device, and which has a compact structure and requires a relatively small installation space.

According to one aspect of the present application, a slip ring assembly is provided and includes: a base body, at least one first conductor, and at least one second conductor. The base body is configured to have an annular shape and adapted to be made of an insulating material, and the base body includes a first side and a second side opposite to the first side along an axial direction of the base body. The first conductor is configured to have an annular shape and adapted to be fixed on the first side or the second side. The second conductor being configured to have an annular shape and adapted to be fixed on the first side or the second side. The first conductor and the second conductor are electrically insulated from each other, and a projection of the first conductor and a projection of the second conductor along the axial direction of the base body at least partially overlap.

In an embodiment, the first conductor and the second conductor are arranged on the same side of the base body; a first insulator is arranged between the first conductor and the second conductor; and the first insulator is adapted such that the first conductor and the second conductor are electrically insulated from each other.

In an embodiment, the slip ring assembly further includes at least one third conductor, wherein the third conductor is configured to have an annular shape and is adapted to be fixed on the base body, and the third conductor, the first conductor, and the second conductor are arranged on the same side of the base body.

In an embodiment, an annular groove is formed on the base body.

In an embodiment, the first conductor or the third conductor is arranged in the annular groove.

In an embodiment, a plurality of first electrically isolating slots are formed in a portion of the first conductor, whose projection does not overlap the projection of the second conductor along the axial direction of the base body; and the plurality of first electrically isolating slots are spaced apart from each other along a circumferential direction of the first conductor.

In an embodiment, the plurality of first electrically isolating slots are arranged at equal intervals in the circumferential direction of the first conductor.

In an embodiment, each of the plurality of first electrically isolating slots is configured to extend in a radial direction of the first conductor.

In an embodiment, a plurality of second electrically isolating slots are formed in a portion of the third conductor, whose projection does not overlap the projection of the first conductor in the axial direction of the base body; the plurality of second electrically isolating slots are spaced apart from each other along a circumferential direction of the third conductor.

In an embodiment, the plurality of second electrically isolating slots are arranged at equal intervals in the circumferential direction of the third conductor.

In an embodiment, each of the plurality of second electrically isolating slots is configured to extend in a radial direction of the third conductor.

In an embodiment, the first conductor is arranged on the first side of the base body; the second conductor is arranged on the second side of the base body; a conductor group includes at least one first conductor and at least one second conductor; and the slip ring assembly includes at least one conductor group.

In an embodiment, each conductor group includes one first conductor and one second conductor, and the first conductor and the second conductor are configured such that the projection of the first conductor and the projection of the second conductor along the axial direction of the base body completely overlap.

In an embodiment, each conductor group includes one first conductor and at least two second conductors; the projection of the first conductor at least partially overlap projections of the at least two second conductors in the axial direction of the base body, respectively; and the at least two second conductors are configured to be electrically insulated from each other.

In an embodiment, the number of the first conductor is different from the number of the second conductor.

In an embodiment, a projection of each first conductor along the axial direction of the base body at least partially overlaps a projection of one second conductor along the axial direction of the base body.

In an embodiment, the slip ring assembly further includes a first brush and a second brush, wherein the first brush is adapted to form an electrical contact with the first conductor, and the second brush is adapted to form an electrical contact with the second conductor.

In an embodiment, the slip ring assembly further includes a third brush, wherein the third brush is adapted to form an electrical contact with the third conductor.

According to another aspect of the invention, a medical device is provided and includes a treatment device. The treatment device includes any slip ring assembly in the embodiments above, and the slip ring assembly is adapted to act as a power supply medium or a communication medium of the treatment device.

In an embodiment, the medical device further includes a magnetic resonance imaging device.

### BRIEF DESCRIPTION OF THE DRAWINGS

The above and other objectives, features and advantages of the present invention will become more apparent through a more particular description of the preferred embodiments of the present invention as shown in the accompanying drawings. The same reference numerals refer to the same parts throughout the drawings. The drawings are not intentionally drawn to scale to actual size, but intended to emphasize and illustrate the subject of the present invention.

Other features, objectives and advantages of the present invention will become more apparent upon reading the detailed description of the non-limiting embodiments with reference to the following drawings:

FIG. 1 is a perspective diagram of a medical device according to an embodiment of the present application;

FIG. 2 is a schematic plan view of a slip ring assembly in the prior art;

FIG. 3 is a schematic plan view of a slip ring assembly according to an embodiment of the present application;

FIG. 4 is a perspective diagram of the slip ring assembly according to an embodiment of the present application;

FIG. 5 is a schematic sectional view of the slip ring assembly shown in FIG. 4;

FIG. 6 is a perspective diagram of the slip ring assembly according to an embodiment of the present application;

FIG. 7 is a schematic sectional view of the slip ring assembly shown in FIG. 6;

FIG. 8 is a schematic sectional view of the slip ring assembly according to an embodiment of the present application;

FIG. 9 is a perspective diagram of the slip ring assembly according to an embodiment of the present application;

FIG. 10 is a schematic sectional view of the slip ring assembly shown in FIG. 9;

FIG. 11 is a perspective diagram of the slip ring assembly according to an embodiment of the present application;

FIG. 12 is a schematic sectional view of the slip ring assembly shown in FIG. 11;

FIG. 13 is a perspective diagram of the slip ring assembly according to an embodiment of the present application;

FIG. 14 is a schematic sectional view of the slip ring assembly shown in FIG. 13;

FIG. 15 is a schematic sectional view of the slip ring assembly according to an embodiment of the present application;

FIG. 16 is a schematic sectional view of the slip ring assembly according to an embodiment of the present application; and

FIG. 17 is a schematic sectional view of the slip ring assembly according to an embodiment of the present application.

### DETAILED DESCRIPTION OF THE EMBODIMENTS

In order to facilitate understanding of the present application, the present application will be described more completely hereinafter with reference to the relevant drawings. Preferred embodiments of the present application are shown in the accompanying drawings, however, the present application may be implemented in many different forms and is not limited to the embodiments described herein. Rather, these embodiments are provided so as to make the disclosure of the present application more thorough and complete.

It should be noted that, when an element is referred to as “connected” to another element, it may be directly connected to and integral with the other element, or there may be intervening elements therebetween. The terms “installation”, “one end”, “another end”, and similar expressions used herein are for illustrative purposes only.

Unless otherwise defined, all technical and scientific terms used herein have the same meaning as commonly understood by one of the ordinary skilled in the technical field of this application. The terminology used herein in the description of the application is for the purpose of describing specific embodiments only and is not intended to limit the application. The term “and/or” herein includes any and all combinations of one or more of the associated listed items.

In order to make the purpose, technical solutions and advantages of the present application clearer, the present application will be further described in detail hereinafter with reference to the drawings and embodiments. It should be understood that the specific embodiments described herein are only used to explain the present application, but not intended to limit the present application.

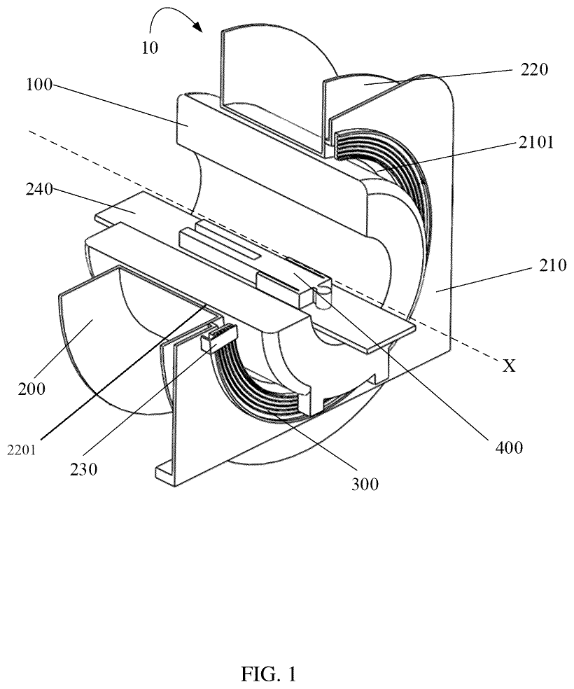

Referring to FIG. 1, FIG. 1 is a perspective diagram of a medical device 10 according to an embodiment of the present application. The medical device 10 includes: a magnetic resonance imaging (MRI) device 100, a treatment device 200, and a bed assembly 240. The MRI device 100 is configured to be in a cylindrical shape and have a hollow portion. The treatment device 200 is, for example, a linear accelerator 200. Optionally, the treatment device 200 may also be any other suitable treatment device, which is not limited in this application. The linear accelerator 200 includes: an accelerator stator 210, an accelerator roller 220, and an electrically connecting terminal 230. The accelerator stator 210 is fixedly arranged, and the accelerator stator 210 has a circular first opening 2101. The accelerator roller 220 has a circular second opening 2201, and the second opening 2201 and the first opening 2101 have a common axis X. The accelerator roller 220 is configured to be pivotable around the axis X relative to the accelerator stator 210. The slip ring assembly 300 is provided on one side of the accelerator roller 220, and the slip ring assembly 300 is arranged coaxially with the first opening 2101 and the second opening 2201. The electrically connecting terminal 230 is disposed on the accelerator stator 210 and may be electrically connected to the slip ring assembly 300. In this embodiment, the electrically connecting terminal 230 is, for example, a carbon brush 230. Optionally, the electrically connecting terminal 230 may also be any other suitable electrical connection device, such as a metal brush, etc., which is not limited in the application, as long as it may be ensured that the slip ring assembly 300 is electrically connected to the linear accelerator 200. In this embodiment, the slip ring assembly 300 is arranged on the accelerator roller 220, and the electrically connecting terminal 230 is arranged on the accelerator stator 210. When the linear accelerator 200 operates, the slip ring assembly 300 pivots together with the accelerator roller 220, and the slip ring assembly 300 is electrically connected to the linear accelerator 200 by the carbon brush 230. Those skilled in the art may understand that the slip ring assembly 300 and the electrically connecting terminal 230 may also be arranged at other suitable positions, as long as it may be achieved that the slip ring assembly 300 and the electrically connecting terminal 230 may slide relative to each other, and that the slip ring assembly 300 may be electrically connected to the linear accelerator 200 by the carbon brush 230. The position of the slip ring assembly 300 and the position of the electrically connecting terminal 230 are not limited in the present application. For example, optionally, the slip ring assembly 300 may also be arranged on the accelerator stator 210, and the electrically connecting terminal 230 is arranged on the accelerator roller 220. When the linear accelerator 200 operates, the carbon brush 230 pivots together with the accelerator roller 220, and the slip ring assembly 300 is electrically connected to the linear accelerator 200 by the carbon brush 230. The bed assembly 240 is configured to be capable of at least partially moving into the hollow portion of the MRI device 100, and includes a bed board adapted to carry a patient 400 to be examined or to be treated. When the medical device 10 is in operation, the patient 400 is positioned on the bed board of the bed assembly 240, and the MRI device 100 is adapted to perform MRI on the region of interest (ROI) of the patient 400, to obtain magnetic resonance images. The linear accelerator 200 is adapted to perform a radiotherapy on the lesion tissue within the ROI based on the magnetic resonance images. Preferably, the MRI device 100 can perform the MRI on the tissue region of interest at any time, such as before, during, or after a radiotherapy. Preferably, the linear accelerator 200 can perform the radiotherapy on the lesion tissue within the ROI based on fusion images formed by the MRI images and images of other types, thereby further improving the accuracy of the radiotherapy.

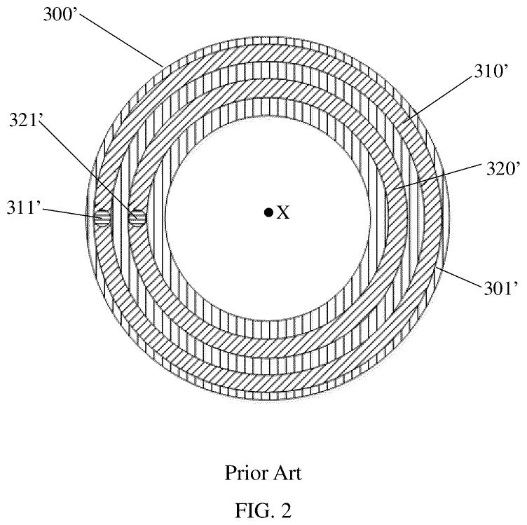

Referring to FIG. 2, FIG. 2 is a schematic plan view of the slip ring assembly 300′ in the prior art. The slip ring assembly 300′ includes: a base body 301′, a first conductor 310′, and a second conductor 320′. The first conductor 310′ and the second conductor 320′ are coaxially arranged on the same side of the base body 301′, and a first brush 311′ and a second brush 321′ are arranged on one side of the first conductor 310′ and on one side of the second conductor 320′ respectively away from the base body 301′. When the slip ring assembly 300′ is powered on, the magnetic fields generated by the first conductor 310′ and the second conductor 320′ will interact with the central magnetic field of the MRI device, which will interfere with a normal operation of the MRI device, thus reducing the accuracy of MRI.

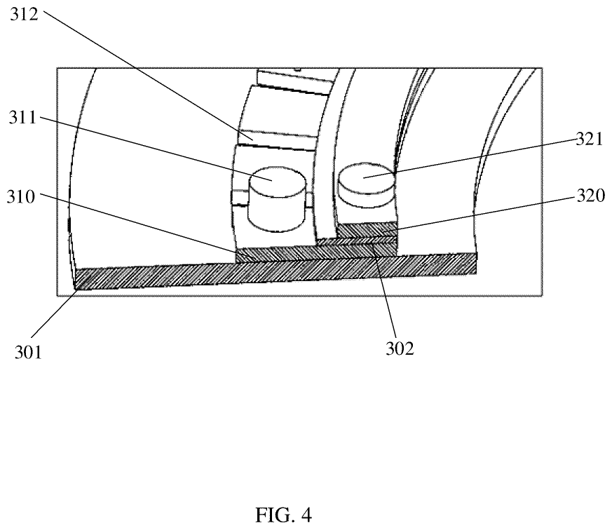

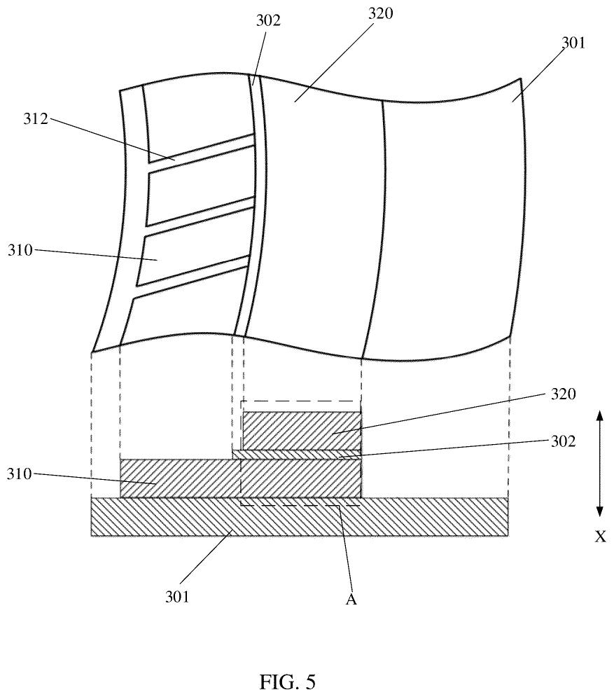

Referring to FIG. 1, FIG. 4 and FIG. 5, FIG. 4 is a perspective diagram of the slip ring assembly according to an embodiment of the present application, and FIG. 5 is a schematic sectional view of the slip ring assembly shown in FIG. 4. In the embodiment shown in FIG. 4 to FIG. 5, the slip ring assembly 300 includes: a base body 301, at least one first conductor 310, and at least one second conductor 320. The base body 301 is configured as an annular sheet structure and is adapted to be made of an insulating material. In the axial direction of the base body 301, the base body 301 includes a first side, and a second side opposite to the first side. The first side may be the side away from the accelerator roller 220 or the side facing the accelerator roller 220. The first conductor 310 is configured as an annular sheet structure, and the second conductor 320 is configured as an annular sheet structure. The first conductor 310 and the second conductor 320 are electrically insulated from each other. The projections of the first conductor 310 and second conductor 320 along the axial direction of the base body 301 (for example, the direction parallel to the X axis) at least partially overlap, that is, the projections have an overlapped area A.

In the embodiment shown in FIG. 4 to FIG. 5, the first conductor 310 and the second conductor 320 are arranged on the same side of the base body 301, such as on the first side or the second side. Optionally, the first conductor 310 and the second conductor 320 may also be arranged in other manners, for example, the first conductor 310 and the second conductor 320 are arranged on different sides of the base body 301, which is not limited in this application, as long as it is satisfied that the projections of the first conductor 310 and second conductor 320 along the axial direction of the base body 301 at least partially overlap.

In the embodiment shown in FIG. 4 to FIG. 5, the base body 301, the first conductor 310 and the second conductor 320 are coaxially arranged. Optionally, the first conductor 310 and the second conductor 320 may also be arranged in other manners, for example, the base body 301, the first conductor 310, and the second conductor 320 are arranged non-axially, which is not limited in this application, as long as it is satisfied that the projections of the first conductor 310 and second conductor 320 along the axial direction of the base body 301 at least partially overlap.

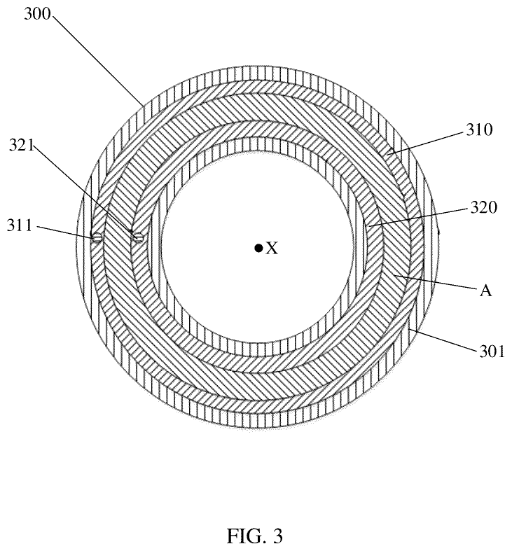

In the embodiment shown in FIG. 4 and FIG. 5, the first conductor 310 and the second conductor 320 have the same inner diameter, and an outer diameter of the first conductor 310 is greater than an outer diameter of the second conductor 320. In this case, the overlapped area of the projections of the first conductor 310 and second conductor 320 along the axial direction is equal to the area of the projection of the second conductor 320 along the axial direction. Optionally, the first conductor 310 and the second conductor 320 may have the same inner diameter and the same outer diameter, or have different inner diameters and different outer diameters, or have the same inner diameter and different outer diameters, or have different inner diameters and the same outer diameter, as long as it is satisfied that when the side of the base body 301 where the first conductor 310 and the second conductor 320 are arranged is viewed along the axial direction of the base body 301, the first conductor 310 and the second conductor 320 may be seen at the same time, that is, the projections of the first conductor 310 and second conductor 320 at least partially overlap along the axial direction of the base body 3. For example, referring to FIG. 3, in the embodiment shown in FIG. 3, the first conductor 310 and the second conductor 320 may have different inner diameters and different outer diameters, and the projections of the first conductor 310 and second conductor 320 along the axial direction of the base body 301 have the overlapped area A.

In the slip ring assembly 300 according to this embodiment, the base body 301, the first conductor 310, and the second conductor 320 are all configured as the annular sheet structures, and the structure of the slip ring assembly 300 in the axial direction (namely an X-direction shown in FIG. 5) is very compact, so the slip ring assembly 300 requires less axial installation space. When the slip ring assembly 300 according to this embodiment is powered on, since the projections of the first conductor 310 and the second conductor 320 along the axial direction of the base body 301 at least partially overlap, the magnetic fields generated by the currents flowing through the portion of the first conductor 310 and the portion of the second conductor 320, which correspond to the overlapped area A respectively, at least partially offset each other, thereby reducing the interference magnetic field generated in the slip ring assembly 300, further reducing the interference in the central magnetic field of the MRI device, and optimizing the accuracy of the MRI.

Continuing to refer to FIG. 4 to FIG. 5, in this embodiment, a first insulator 302 is arranged between the first conductor 310 and the second conductor 320, so that the first conductor 310 and the second conductor 320 are electrically insulated from each other. Specifically, the first insulator 302 may be in an annular sheet shape, and the area of the projection of the first insulator 302 along the axial direction of the base body 301 is greater than or equal to the overlapped area of the first conductor 310 and the second conductor 320 along the axial direction.

Continuing to refer to FIG. 4 and FIG. 5, multiple first electrically isolating slots 312 are further formed in a portion of the first conductor 310, whose projection does not overlap the projection of the second conductor 320 in the axial direction of the base body 301. In the portion of the first conductor 310 where the first electrically isolating slot 312 is formed, the current cannot flow in a circumferential direction of the first conductor 310. In this embodiment, each first electrically isolating slot 312 is configured to extend linearly in the first conductor 310, and the plurality of first electrically isolating slots 312 are spaced apart from each other in the circumferential direction of the first conductor 310. Those skilled in the art should understand that the first electrically isolating slots 312 each may also have other shapes and arrangements, which are not limited in the application, as long as it may be satisfied that the current in the portion of the first conductor 310, where the first electrically isolating slots 312 are formed, cannot flow in the circumferential direction of the first conductor 310. Optionally, each of the first electrically isolating slots 312 may be configured to extend in the first conductor 310 in a curved or zigzag manner. Optionally, the plurality of first electrically isolating slots 312 are configured to extend from an outer edge of the portion of the first conductor 310 not overlapped by the second conductor 320, to an inner edge of the portion of the first conductor 310 not overlapped by the second conductor 320. Optionally, the plurality of first electrically isolating slots 312 are arranged at equal intervals in the circumferential direction of the first conductor 310. Optionally, the plurality of first electrically isolating slots 312 are configured to extend in the radial direction of the first conductor 310.

In this embodiment, the slip ring assembly 300 further includes at least one first brush 311 and at least one second brush 321. The first brush 311 is adapted to form an electrical contact with the first conductor 310. The second brush 321 is adapted to form an electrical contact with the second conductor 320. In this embodiment, the first brush 311 and the second brush 321 are arranged on the sides of the first conductor 310 and second conductor 320 respectively away from the base body 301. Those skilled in the art should understand that the first brush 311 and the second brush 321 may also be arranged at other suitable positions, as long as they can meet the requirements for forming the electrical contacts with the first conductor 310 and the second conductor 320 respectively. Optionally, the first brush 311 is disposed on a radial-direction inner surface or a radial-direction outer surface of the first conductor 310. Optionally, the second brush 321 is disposed on a radial-direction inner surface or a radial-direction outer surface of the second conductor 320.

Further, in another embodiment, an annular groove may also be formed on the first side of the base body 301, and the first conductor 310 is arranged in the annular groove. Preferably, a depth of the annular groove is equal to a thickness of the first conductor 310.

In the embodiment shown in FIG. 4 to FIG. 5, the number of the first conductor 310 and the number of second conductor 320 may be the same or different. The projection of each first conductor 310 along the axial direction of the base body 301 at least partially overlaps the projection of the second conductor 320 along the axial direction of the base body 301. A conductor group may include at least one first conductor 310 and at least one second conductor 320, and the slip ring assembly 300 includes at least one conductor group. Those skilled in the art should understand that the number of the first conductor 310 and the number of the second conductor 320 in each conductor group, and the number of the conductor group in the slip ring assembly 300 may be selected according to actual needs. For example, in an embodiment, the conductor group includes one first conductor 310 and one second conductor 320, and the slip ring assembly 300 includes one conductor group. For example, in another embodiment, the conductor group includes two first conductors 310 and one second conductor 320, and the slip ring assembly 300 includes two conductor groups.

In the slip ring assembly 300 according to this embodiment, the base body 301, the first conductor 310, and the second conductor 320 are all configured as the annular sheet structures, and the structure of the slip ring assembly 300 in the axial direction is very compact, so the slip ring assembly 300 requires less axial installation space. When the slip ring assembly 300 according to this embodiment is powered on, since the projections of the first conductor 310 and the second conductor 320 along the axial direction of the base body 301 at least partially overlap, the magnetic fields generated by the currents flowing through the portions of the first conductor 310 and the second conductor 320 corresponding to the overlapped area A respectively at least partially offset each other, thereby reducing the interference magnetic field generated in the slip ring assembly 300, thereby further reducing the interference in the central magnetic field of the MRI device. Furthermore, since the plurality of first electrically isolating slots 312 are formed in the first conductor 310, the current can only flow through the portions of the first conductor 310 and second conductor 320 corresponding to the overlapped area A respectively, thereby maximizing the reduction of the current-loop area of the slip ring assembly 300, further reducing the interference magnetic field generated in the slip ring assembly 300, and further reducing the interference in the central magnetic field of the MRI device, and ensuring the accuracy of MRI.

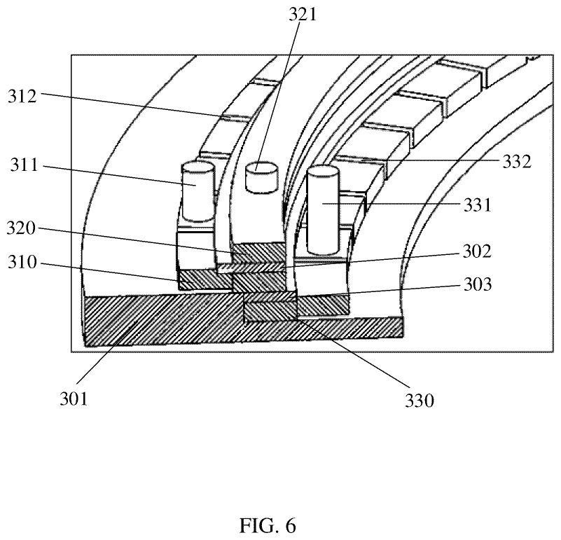

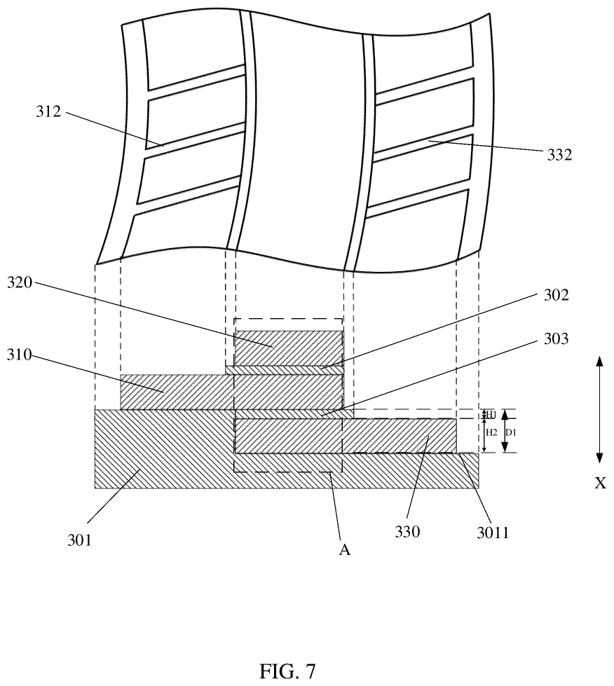

Referring to FIGS. 1, 6, and 7, FIG. 6 is a perspective diagram of the slip ring assembly 300 according to an embodiment of the present application. FIG. 7 is a schematic sectional view of the slip ring assembly 300 shown in FIG. 6. In the embodiment shown in FIG. 6 and FIG. 7, the structural and positional relationships of the first conductor 310 and the second conductor 320 are similar to those of the first conductor 310 and the second conductor 320 in the embodiments shown in FIG. 3 to FIG. 5. In this embodiment, the annular assembly 300 further includes at least one third conductor 330. The third conductor 330 is configured as an annular sheet structure and is adapted to be fixed on the base body 301. The third conductor 330, the first conductor 310, and the second conductor 320 are arranged on the same side of the base body 301. The third conductor 330 is electrically insulated from the first conductor 310 or from the second conductor 320.

Optionally, the third conductor 330 is arranged between the first conductor 310 and the base body 301 along the axial direction of the base body 301, as shown in FIG. 7. A second insulator 303 is arranged between the third conductor 330 and the first conductor 310, and the second insulator 303 is adapted to electrically insulate the third conductor 330 from the first conductor 310. The third conductor 330 and the first conductor 310 at least partially overlap along the axial direction of the base body 301, such that the first conductor 310, the second conductor 320, and the third conductor 330 form an overlapped area A along the axial direction of the base body 301.

Optionally, the third conductor 330 is arranged on one side of the second conductor 320 along the axial direction of the base body 301, and a third insulator is arranged between the third conductor 330 and the second conductor 320. The third insulator is adapted to electrically insulate the third conductor 330 from the second conductor 320. The third conductor 330 and the second conductor 320 at least partially overlap along the axial direction of the base body 301, such that the first conductor 310, the second conductor 320, and the third conductor 330 form the overlapped area along the axial direction of the base body 301.

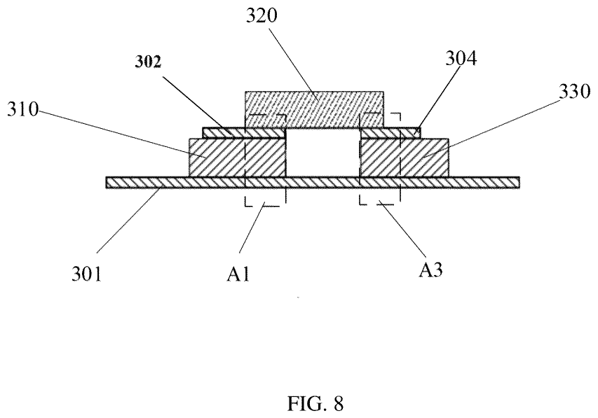

Optionally, the third conductor 330 is arranged between the second conductor 320 and the base body 301 along the axial direction of the base body 301. Referring to FIG. 8, a schematic sectional view of the slip ring assembly 330 according to an embodiment of the present application is shown. In the embodiment shown in FIG. 8, the first conductor 310 and the third conductor 330 are arranged on the same side of the base body 301, and the second conductor 320 is arranged on the same side of the first conductor 310 and third conductor 330 along the axial direction of the base body 301. The first insulator 302 is arranged between the first conductor 310 and the second conductor 320, so that the first conductor 310 and the second conductor 320 are electrically insulated from each other. A third insulator 304 is arranged between the third conductor 330 and the second conductor 320, so that the third conductor 330 and the second conductor 320 are electrically insulated from each other. The projections of the first conductor 310 and second conductor 320 along the axial direction of the base body 301 at least partially overlap to form an overlapped area A1, the projections of the third conductor 330 and second conductor 320 along the axial direction of the base body 301 at least partially overlap to form an overlapped area A3. Optionally, electrically isolating slots are formed in a portion of the first conductor 310, whose projection does not overlap the projection of the second conductor 320 in the axial direction of the base body 301. Optionally, electrically isolating slots are formed in a portion of the third conductor 330, whose projection does not overlap the projection of the second conductor 320 in the axial direction of the base body 301. Optionally, electrically isolating slots are formed in a portion of the second conductor 320, whose projection does not overlap the projections of the first conductor 310 and third conductor 330 in the axial direction of the base body 301.

In the embodiment shown in FIG. 6 and FIG. 7, an annular groove 3011 is further formed on the first side of the base body 301, and the third conductor 330 is arranged in the annular groove 3011. Optionally, the first conductor 310 is arranged in the annular groove 3011.

In the embodiment shown in FIG. 6 and FIG. 7, the second insulator 303 is configured as an annular sheet structure, and the area of the projection of the second insulator 303 along the axial direction of the base body 301 is greater than or equal to the overlapped area of the third conductor 330 and first conductor 310 along the axial direction. Optionally, a sum of a thickness H1 of the second insulator 303 and a thickness H2 of the third conductor 330 is equal to a depth D1 of the annular groove 3011.

In the embodiment that the first conductor 310 is arranged in the annular groove 3011, the third conductor 330 is arranged over the second conductor 320, and the second insulator 303 is arranged between the third conductor 330 and the second conductor 320. The second insulator 303 is adapted to electrically insulate the third conductor 330 from the second conductor 320. The third conductor 330 and the second conductor 310 at least partially overlap in the axial direction of the base body 301. Optionally, the sum of the thickness of the second insulator 303 and the thickness of the first conductor 310 is equal to the depth of the annular groove 3011.

In the slip ring assembly 300 of the embodiment shown in FIG. 6 and FIG. 7, the base body 301, the first conductor 310, the second conductor 320, and the third conductor 330 are all configured as the annular sheet structures, and the structure of the slip ring assembly 300 in the axial direction (namely an X-direction shown in FIG. 7) is very compact, so the slip ring assembly 300 requires less axial installation space. When the slip ring assembly 300 according to this embodiment is powered on, since the projections of the first conductor 310, second conductor 320 and third conductor 330 along the axial direction of the base body 301 at least partially overlap, the magnetic fields generated by the currents flowing through the portions of the first conductor 310, second conductor 320 and third conductor 330 corresponding to the overlapped area A respectively at least partially offset each other, thereby reducing the interference magnetic field generated in the slip ring assembly 300, thereby further reducing the interference in the central magnetic field of the MRI device, and optimizing the accuracy of the MRI.

Continuing to refer to FIG. 7, in this embodiment, a plurality of first electrically isolating slots 312 are formed in the portion of the first conductor 310 without being overlapped by the second conductor 320 in the axial direction of the base body 301, and a plurality of second electrically isolating slots 332 are formed in the portion of the third conductor 330 without being overlapped by the first conductor 310 in the axial direction of the base body 301. In the portions of the first conductor 310 and third conductor 330 where the isolation slots are formed, currents cannot flow in the circumferential directions of the first conductor 310 and third conductor 330 respectively. In this embodiment, each of the first electrically isolating slots 312 is configured to extend linearly in the first conductor 310, and each of the second electrically isolating slots 332 is configured to extend linearly in the third conductor 330. The plurality of first electrically isolating slots 312 are spaced apart from each other in the circumferential direction of the first conductor 310, and the plurality of third electrically isolating slots 332 are spaced apart from each other in the circumferential direction of the third conductor 310. Those skilled in the art should understand that the first electrically isolating slots 312 and the third electrically isolating slots 332 may have other shapes and arrangements, which are not limited in the application, as long as it may be satisfied that the currents in the portions of the first conductor 310 and third conductor 330, where the first electrically isolating slots 312 and the third electrically isolating slots 332 are formed respectively, cannot flow in the circumferential directions of the first conductor 310 and third conductor 330 respectively. Optionally, each of the first electrically isolating slots 312 may be configured to extend in the first conductor 310 in a curved or zigzag manner. Optionally, each of the third electrically isolating slots 332 may be configured to extend in the third conductor 330 in a curved or zigzag manner. Optionally, the plurality of first electrically isolating slots 312 are configured to extend from the outer edge of the portion of the first conductor 310, whose projection does not overlap the projection of the second conductor 320 in the axial direction of the base body 301, to the inner edge of the portion of the first conductor 310, whose projection does not overlap the projection of the second conductor 320 in the axial direction of the base body 301. Optionally, the plurality of third electrically isolating slots 332 are configured to extend from the outer edge of the portion of the third conductor 330, whose projection does not overlap the projection of the second conductor 320 in the axial direction of the base body 301 to the inner edge of the portion of the third conductor 330, whose projection does not overlap the projection of the second conductor 320 in the axial direction of the base body 301. Optionally, the plurality of first electrically isolating slots 312 are arranged at equal intervals in the circumferential direction of the first conductor 310. Optionally, the plurality of second electrically isolating slots 332 are arranged at equal intervals in the circumferential direction of the third conductor 330. Optionally, the plurality of first electrically isolating slots 312 each are configured to extend in the radial direction of the first conductor 310. Optionally, the plurality of third electrically isolating slots 332 each are configured to extend in the radial direction of the third conductor 330.

In the embodiment shown in FIG. 6 and FIG. 7, the slip ring assembly further includes a third brush 331 adapted to form an electrical contact with the third conductor 330. The third brush 331 is adapted to form the electrical contact with the third conductor 330. In this embodiment, the third brush 331 is arranged on a side of the third conductor 330 away from the base body 301. Those skilled in the art should understand that the third brush 331 may also be arranged at other suitable positions, as long as it may be satisfied that the third brush forms the electrical contact with the third conductor 330. Optionally, the third brush 331 is arranged on the radial-direction inner surface or the radial-direction outer surface of the third conductor 330.

In the embodiment shown in FIG. 6 and FIG. 7, the numbers of the first conductor 310, second conductor 320 and third conductor 330 may be the same or different. At least one first conductor 310, at least one second conductor 320, and at least one third conductor 330 may form a conductor group, and the slip ring assembly 300 includes at least one conductor group. Those skilled in the art should understand that the numbers of the first conductor 310, second conductor 320 and third conductor 330 in each conductor group and the number of the conductor groups in the slip ring assembly 300 may be selected according to actual needs. For example, in an embodiment, the conductor group includes one first conductor 310, one second conductor 320, and one third conductor 330, and the slip ring assembly 300 includes one conductor group. In the radial direction of the base body 301, the diameters of the annular conductors in each annular conductor group gradually increase or decrease.

In the slip ring assembly 300 according to this embodiment, the base body 301, the first conductor 310, the second conductor 320, and the third conductor 330 are all configured as the annular sheet structures, and the structure of the slip ring assembly 300 in the axial direction is very compact, so the slip ring assembly 300 requires less axial installation space. When the slip ring assembly 300 according to this embodiment is powered on, since the projections of the first conductor 310, second conductor 320 and third conductor 330 along the axial direction of the base body 301 at least partially overlap, the magnetic fields generated by the current flowing through the portions of the first conductor 310, second conductor 320 and third conductor 330 corresponding to the overlapped area A respectively at least partially offset each other, thereby reducing the interference magnetic field generated in the slip ring assembly 300, and further reducing the interference in the central magnetic field of the MRI device. Furthermore, since the plurality of first electrically isolating slots 312 and the plurality of second electrically isolating slots 332 are formed in the first conductor 310 and the third conductor 330, respectively, the currents can only flow through the portions of the first conductor 310, second conductor 320 and third conductor 330 corresponding to the overlapped area A respectively, thereby maximizing the reduction of the current-loop area of the slip ring assembly 300, further reducing the interference magnetic field generated in the slip ring assembly 300, and thereby further reducing the interference in the central magnetic field of the MRI device, and ensuring the accuracy of the MRI.

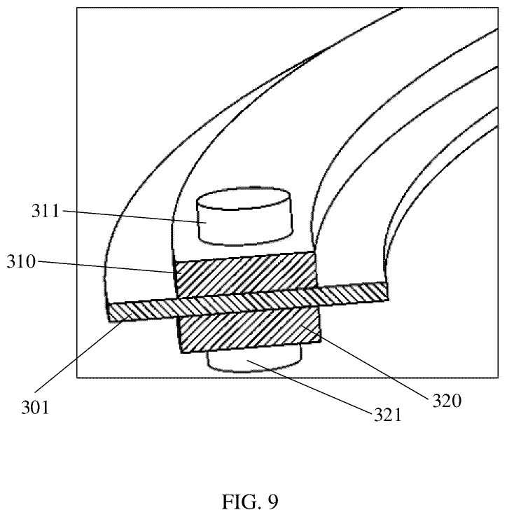

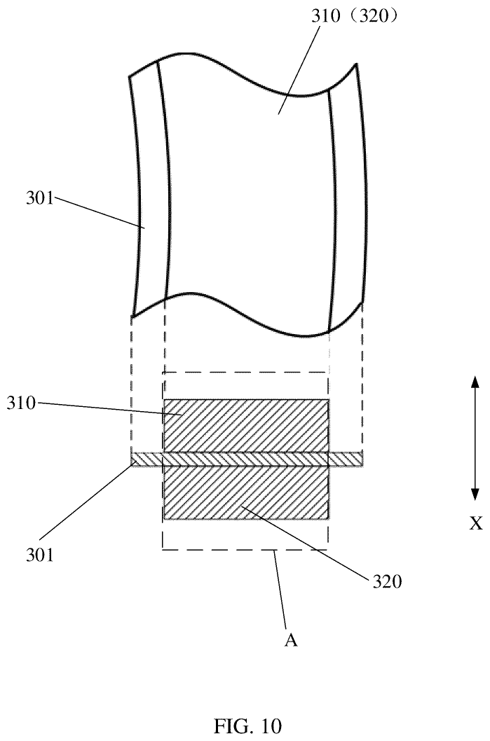

Referring to FIG. 9 and FIG. 10, FIG. 9 is a perspective diagram of the slip ring assembly according to an embodiment of the present application, and FIG. 10 is a schematic sectional view of the slip ring assembly shown in FIG. 9. In the embodiment shown in FIG. 9 and FIG. 10, the first conductor 310, the second conductor 320, the first brush 311 and the second brush 321 are similar to the embodiments shown in FIG. 3 to FIG. 5, and differ from the embodiments shown in FIG. 3 to FIG. 5 in that at least one first conductor 310 and at least one second conductor 320 are arranged on different sides of the base body 301 respectively.

In the embodiment shown in FIG. 9 and FIG. 10, the number of first conductor 310 and the number of the second conductor 320 may be the same or different. The conductor group includes at least one first conductor 310 and at least one second conductor 320, and the slip ring assembly 300 includes at least one conductor group. Those skilled in the art should understand that the numbers of the first conductor 310 and second conductor 320 in each conductor group and the number of conductor groups in the slip ring assembly 300 may be selected according to actual needs.

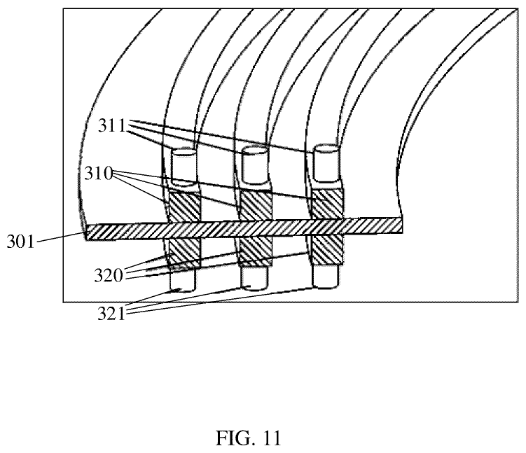

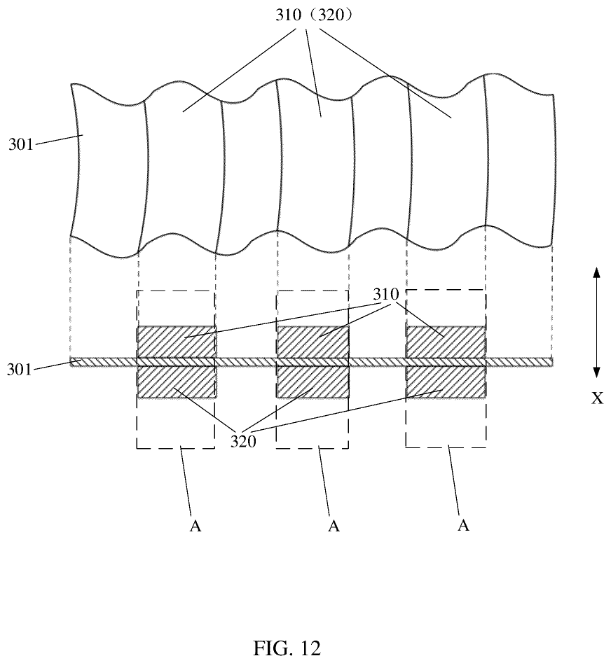

Optionally, in the embodiments shown in FIG. 11 and FIG. 12, the conductor group includes one first conductor 310 and one second conductor 320, and the slip ring assembly 300 includes three conductor groups. In each conductor group, the projections of the first conductor 310 and the second conductor 320 in the axial direction of the base body 301 at least partially overlap. Optionally, the slip ring assembly 300 may include another suitable number of conductor groups, such as one conductor group, two conductor groups, or four conductor groups, etc.

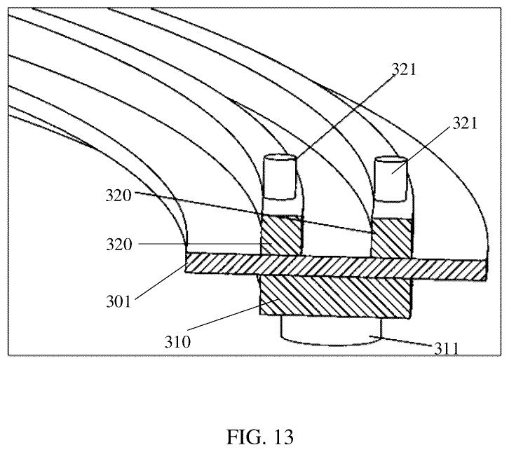

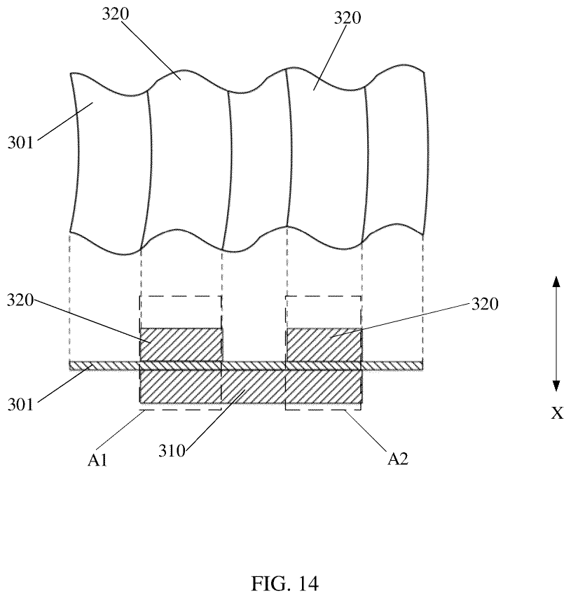

Optionally, in the embodiment shown in FIG. 13 and FIG. 14, the conductor group includes one first conductor 310 and two second conductors 320, and the slip ring assembly 300 includes one conductor group. In the conductor group, the projection of the first conductor 310 at least partially overlaps the projections of the two second conductors 320 in the axial direction of the base body 301, respectively. Optionally, the slip ring assembly 300 may include another suitable number of conductor groups, such as two conductor groups, three conductor groups, or four conductor groups, etc.

In the embodiments in FIG. 9 and FIG. 10, FIG. 11 and FIG. 12, and FIG. 13 and FIG. 14, the base body 301, the first conductor 310 and the second conductor 320 of the slip ring assembly 300 are all configured as the annular sheet structures, and the structure of the slip ring assembly 300 in the axial direction of the base body 301 (namely an X-direction shown in FIGS. 10, 12 and 14) is very compact, so the slip ring assembly 300 requires less axial installation space. When the slip ring assembly 300 according to this embodiment is powered on, since the projections of the first conductor 310 and second conductor 320 along the axial direction of the base body 301 at least partially overlap, the magnetic fields generated by the current flowing through the portions of the projections of the first conductor 310 and second conductor 320 corresponding to the overlapped area A respectively at least partially offset each other, thereby reducing the interference magnetic field generated in the slip ring assembly 300, further reducing the interference in the central magnetic field of the MRI device, and optimizing the accuracy of the MRI.

Further, in the embodiment shown in FIG. 13 and FIG. 14, a plurality of third electrically isolating slots (not shown) are formed in the portion of the first conductor 310 without being overlapped by the second conductor 320 in the axial direction of the base body 301, and the current cannot flow in the circumferential direction through the portion of the first conductor 310, where the third electrically isolating slots are formed. In this embodiment, each of the third electrically isolating slots is configured to extend linearly in the first conductor 310, and the plurality of third electrically isolating slots are spaced apart from each other in the circumferential direction of the first conductor 310. Those skilled in the art should understand that the third electrically isolating slot may also have other shapes and arrangements, which are not limited in the application, as long as it may be satisfied that the current cannot flow in the circumferential direction of the first conductor 310 through the portion of the first conductor 310, where the third electrically isolating slots are formed. Optionally, each third electrically isolating slot is configured to extend in the first conductor 310 in a curved or zigzag manner. Optionally, the plurality of third electrically isolating slots are configured to extend from the outer edge of the portion of the first conductor 310 without being overlapped by the second conductor 320 to the inner edge of the portion of the first conductor 310 without being overlapped by the second conductor 320. Optionally, the plurality of third electrically isolating slots are arranged at equal intervals in the circumferential direction of the first conductor 310. Optionally, the plurality of third electrically isolating slots are configured to extend in the radial direction of the first conductor 310.

Further, in the embodiment shown in FIG. 13 and FIG. 14, the slip ring assembly 330 includes at least two second conductors 320. The two second conductors 320 are arranged on the same side of the base body 301, and the at least two second conductors 320 are electrically insulated from each other. The projection of the first conductor 310 partially overlaps the projections of the two second conductors 320 along the axial direction of the base body 301, respectively, to form a first overlapped area A1 and a second overlapped area A2. Optionally, a gap is formed between the two second conductors 320, so that the at least two second conductors 320 are electrically insulated from each other. Further, insulating material is provided in the gap between the two second conductors 320, so that the at least two second conductors 320 are electrically insulated from each other.

In the slip ring assembly 300 according to this embodiment, the base body 301, the first conductor 310, and the second conductor 320 are all configured as the annular sheet structures, and the structure of the slip ring assembly 300 in the axial direction is very compact, so the slip ring assembly 300 requires less installation space. When the slip ring assembly 300 according to this embodiment is powered on, since the projections of the first conductor 310 and the second conductor 320 along the axial direction of the base body 301 at least partially overlap, the magnetic fields generated by the currents flowing through the portions of the first conductor 310 and the second conductor 320 corresponding to the overlapped area A respectively at least partially offset each other, thereby reducing the interference magnetic field generated in the slip ring assembly 300, further reducing the interference in the central magnetic field of the MRI device. Furthermore, since the plurality of first electrically isolating slots 312 are formed in the first conductor 310, the currents can only flow through the portions of the first conductor 310 and second conductor 320 corresponding to the overlapped area A1 or A2 respectively, thereby maximizing the reduction of the current-loop area of the slip ring assembly 300, further reducing the interference magnetic field generated in the slip ring assembly 300, further reducing the interference in the central magnetic field of the MRI device, and ensuring the accuracy of the MRI.

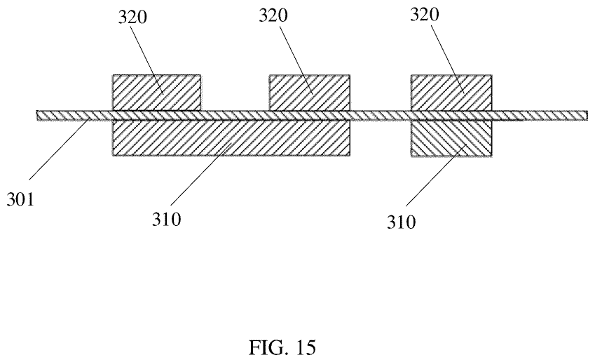

Referring to FIG. 15, FIG. 15 shows a conductor group of the slip ring assembly 300 according to an embodiment of the present invention. The conductor group includes at least two first conductors 310 and at least three second conductors 320. The first conductor 310 and the second conductor 320 are respectively arranged on different sides of the base body 301 in the axial direction. The projections of one first conductor 310 and two of the second conductors 320 along the axial direction of the base body 301 at least partially overlap to obtain an overlapped area. The projection of another first conductor 310 and another second conductor 320 along the axial direction of the base body 301 at least partially overlaps to obtain another overlapped area. In this embodiment, the slip ring assembly 300 includes at least one conductor group. Optionally, the slip ring assembly 300 may include another suitable number of conductor groups, such as two conductor groups, three conductor groups, or four conductor groups, etc.

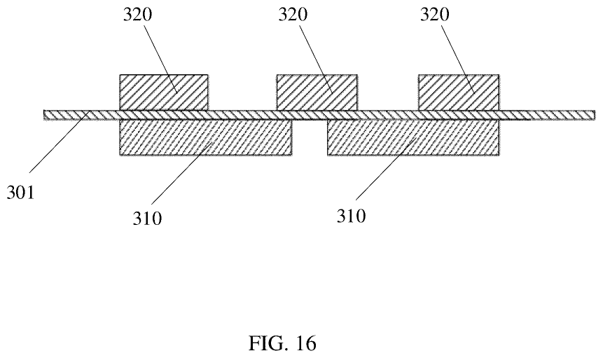

Referring to FIG. 16, FIG. 16 shows a schematic sectional view of the slip ring assembly 300 according to an embodiment of the present application. The conductor group includes at least two first conductors 310 and at least three second conductors 320. The first conductor 310 and the second conductor 320 are arranged on different sides of the base body 301 in the axial direction respectively. The projection of each first conductor 310 and the projections of two of the second conductors 320 along the axial direction of the base body 301 at least partially overlap to obtain an overlapped area. In this embodiment, the slip ring assembly 300 includes at least one conductor group. Optionally, the slip ring assembly 300 may include another suitable number of conductor groups, such as two conductor groups, three conductor groups, or four conductor groups, etc.

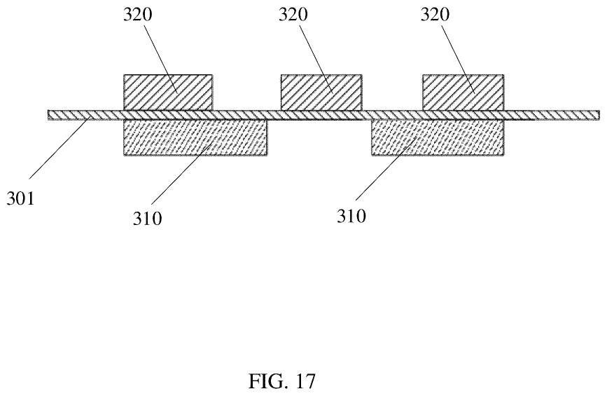

Referring to FIG. 17, FIG. 17 shows the slip ring assembly 300 according to an embodiment of the present application. The conductor group includes at least two first conductors 310 and at least three second conductors 320. The first conductor 310 and the second conductor 320 are arranged on different sides of the base body 301 in the axial direction respectively. The projection of at least one first conductor 310 and the projection of the at least one second conductor 320 along the axial direction of the base body 301 at least partially overlap to obtain an overlapped area, and the projection of at least one second conductor 320 and the projection of any one first conductor 310 in the axial direction of base body 301 do not overlap. In this embodiment, the slip ring assembly 300 includes at least one conductor group. Optionally, the slip ring assembly 300 may include another suitable number of conductor groups, such as two conductor groups, three conductor groups, or four conductor groups, etc.

In the embodiments shown in FIG. 15 to FIG. 17, optionally, electrically isolating slots are formed in the portion of the first conductor 310, whose projection does not overlap the projection of the second conductor 320 along the axial direction of the base body 301. Optionally, electrically isolating slots are formed in the portion of the second conductor 320, whose projection does not overlap the projection of the first conductor 310 in the axial direction of the base body 301.

In the embodiments shown in FIG. 15 to FIG. 17, the base body 301, the first conductor 310, and the second conductor 320 are all configured as the annular sheet structures, and the structure of the slip ring assembly 300 in the axial direction is very compact, so the slip ring assembly 300 requires less installation space. When the slip ring assembly 300 according to this embodiment is powered on, since the projections of the first conductor 310 and second conductor 320 along the axial direction of the base body 301 at least partially overlap, the magnetic fields generated by the currents flowing through the portions of the first conductor 310 and second conductor 320 corresponding to the overlapped area A respectively at least partially offset each other, thereby reducing the interference magnetic field generated in the slip ring assembly 300, further reducing the interference in the central magnetic field of the MRI device. Furthermore, since the plurality of electrically isolating slots are formed in the first conductor 310 or in the second conductor 320, the currents can only flow through the portions of the first conductor 310 and second conductor 320 corresponding to the overlapped area respectively, thereby maximizing the reduction of the current-loop area of the slip ring assembly 300, further reducing the interference magnetic field generated in the slip ring assembly 300, further reducing the interference in the central magnetic field of the MRI device, and ensuring the accuracy of the MRI.

The technical features of the embodiments above may be combined arbitrarily. To make the description concise, not all possible combinations of the technical features in the above embodiments are described. However, as long as there are no contradictions in the combinations of these technical features, all of the combinations should be considered to be within the scope of the specification.

The embodiments above only represent several implementation modes of the present application, and the description thereof is relatively specific and detailed, but it should not be construed as limiting the scope of the patent. It should be noted that for those skilled in the art, various modifications and improvements may be made without departing from the concept of the present application, and all these modifications and improvements belong to the protection scope of the present application. Therefore, the scope of protection of the patent application should be subject to the appended claims.

1. A slip ring assembly, comprising:
a base body, the base body being configured to have an annular shape and adapted to be made of an insulating material, and the base body comprising a first side and a second side opposite to the first side along an axial direction of the base body;
at least one first conductor, the first conductor being configured to have an annular shape and adapted to be fixed on the first side or the second side;
at least one second conductor, the second conductor being configured to have an annular shape and adapted to be fixed on the first side or the second side;
wherein the first conductor and the second conductor are electrically insulated from each other, and a projection of the first conductor and a projection of the second conductor along the axial direction of the base body at least partially overlap.

2. The slip ring assembly according to claim 1, wherein the first conductor and the second conductor are arranged on the same side of the base body; a first insulator is arranged between the first conductor and the second conductor; and the first insulator is adapted such that the first conductor and the second conductor are electrically insulated from each other.

3. The slip ring assembly according to claim 2, further comprising at least one third conductor, wherein the third conductor is configured to have an annular shape and is adapted to be fixed on the base body, and the third conductor, the first conductor, and the second conductor are arranged on the same side of the base body.

4. The slip ring assembly according to claim 1, wherein an annular groove is formed on the base body.

5. The ring assembly according to claim 4, wherein the first conductor or the third conductor is disposed in the annular groove.

6. The slip ring assembly according to claim 1, wherein a plurality of first electrically isolating slots are formed in a portion of the first conductor, whose projection does not overlap the projection of the second conductor along the axial direction of the base body; and the plurality of first electrically isolating slots are spaced apart from each other along a circumferential direction of the first conductor.

7. The slip ring assembly according to claim 6, wherein the plurality of first electrically isolating slots are arranged at equal intervals in the circumferential direction of the first conductor.

8. The slip ring assembly according to claim 6, wherein each of the plurality of first electrically isolating slots is configured to extend in a radial direction of the first conductor.

9. The slip ring assembly according to claim 3, wherein a plurality of second electrically isolating slots are formed in a portion of the third conductor, whose projection does not overlap the projection of the first conductor in the axial direction of the base body; the plurality of second electrically isolating slots are spaced apart from each other along a circumferential direction of the third conductor.

10. The slip ring assembly according to claim 9, wherein the plurality of second electrically isolating slots are arranged at equal intervals in the circumferential direction of the third conductor.

11. The slip ring assembly according to claim 9, wherein each of the plurality of second electrically isolating slots is configured to extend in a radial direction of the third conductor.

12. The slip ring assembly according to claim 1, wherein the first conductor is arranged on the first side of the base body; the second conductor is arranged on the second side of the base body; a conductor group comprises at least one first conductor and at least one second conductor; and the slip ring assembly comprises at least one conductor group.

13. The slip ring assembly according to claim 12, wherein each conductor group comprises one first conductor and one second conductor, and the first conductor and the second conductor are configured such that the projection of the first conductor and the projection of the second conductor along the axial direction of the base body completely overlap.

14. The slip ring assembly according to claim 12, wherein each conductor group comprises one first conductor and at least two second conductors; the projection of the first conductor at least partially overlaps projections of the at least two second conductors in the axial direction of the base body, respectively; and the at least two second conductors are configured to be electrically insulated from each other.

15. The slip ring assembly according to claim 1, wherein the number of the first conductor is different from the number of the second conductor.

16. The slip ring assembly according to claim 1, wherein a projection of each first conductor along the axial direction of the base body at least partially overlaps a projection of one second conductor along the axial direction of the base body.

17. The slip ring assembly according to claim 1, further comprising a first brush and a second brush, wherein the first brush is adapted to form an electrical contact with the first conductor, and the second brush is adapted to form an electrical contact with the second conductor.

18. The slip ring assembly according to claim 3, further comprising a third brush, wherein the third brush is adapted to form an electrical contact with the third conductor.

19. A medical device, comprising a treatment device, characterized in that the treatment device comprises the slip ring assembly according to claim 1, and the slip ring assembly is adapted to act as a power supply medium or a communication medium of the treatment device.

20. The medical device according to claim 19, further comprising a magnetic resonance imaging device.
